(19) United States
(12) Patent Application Publication
(10) Pub. No.: US 2022/0065381 A1
(43) Pub. Date: Mar. 3, 2022

(54) Oxygen Hose System

(71) Applicants: Joseph Nester, Willoughby, OH (US); Thomas Donathan, Willoughby, OH (US)

(72) Inventors: Joseph Nester, Willoughby, OH (US); Thomas Donathan, Willoughby, OH (US)

(21) Appl. No.: 17/011,001

(22) Filed: Sep. 3, 2020

(51) Int. Cl. F16L 57/06; A61M 16/08

(52) U.S. Cl. CPC F16L 57/06; A61M 2202/0208; A61M 16/0875

(57) ABSTRACT

An oxygen hose system for protecting an oxygen hose from tangling and abrasion includes an oxygen hose that is fluidly coupled to an oxygen source for supplying breathing oxygen to a patient. A sleeve is provided and the sleeve is positionable around the oxygen hose. The sleeve is comprised of a resilient material to inhibit the oxygen hose from being chewed by a domesticated animal. Additionally, the sleeve is comprised of a flexible material thereby facilitating the oxygen hose to be routed in various directions.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to hose devices and more particularly pertains to a new hose device for inhibiting an oxygen supply hose from becoming abraded or tangled.

### (2) Description of Related Art Including Information Disclosed Under 37 CFR 1.97 and 1.98

The prior art relates to hose devices including a nasogastric tube that includes a sheath and fastening straps on the sheath to inhibit a patient from removing the nasogastric tube. The prior art discloses a tube that is positionable over a coiled handset cord of a telephone. The prior art discloses a sleeve with ridges thereon that is positionable over a power cord of a power tool. The prior art discloses an oxygen tube that includes a sheath being positioned around an oxygen tube which is attached to a tracheotomy mask. The prior art disclose a sleeve, comprised of a plurality of movable sections, which is positionable around a garden hose to inhibit the garden hose from kinking.

### BRIEF SUMMARY OF THE INVENTION

An embodiment of the disclosure meets the needs presented above by generally comprising an oxygen hose that is fluidly coupled to an oxygen source for supplying breathing oxygen to a patient. A sleeve is provided and the sleeve is positionable around the oxygen hose. The sleeve is comprised of a resilient material to inhibit the oxygen hose from being chewed by a domesticated animal. Additionally, the sleeve is comprised of a flexible material thereby facilitating the oxygen hose to be routed in various directions.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of sleeve of an oxygen hose system according to an embodiment of the disclosure.

FIG. 2 is a front view of a sleeve of an embodiment of the disclosure.

FIG. 3 is a front view of an embodiment of the disclosure.

FIG. 4 is a cross sectional view taken along line 4-4 of FIG. 3 of an embodiment of the disclosure.

### DETAILED DESCRIPTION OF THE INVENTION

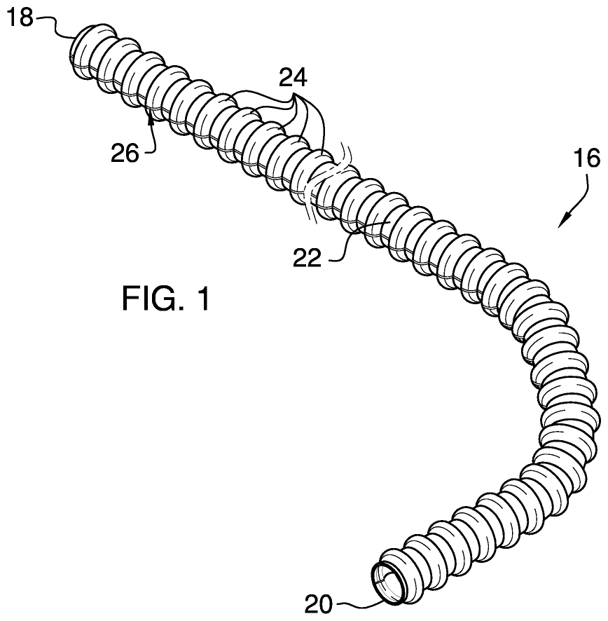

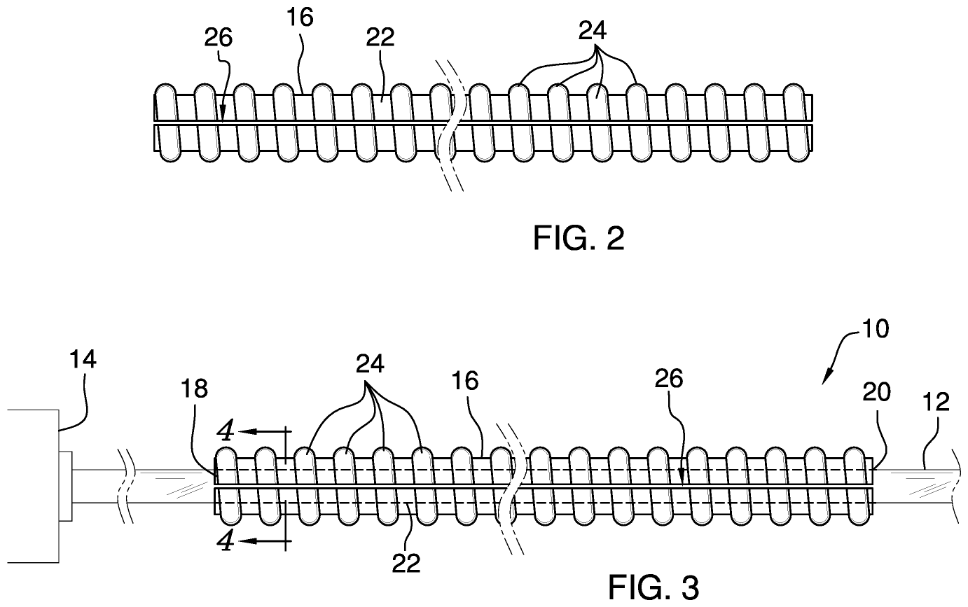

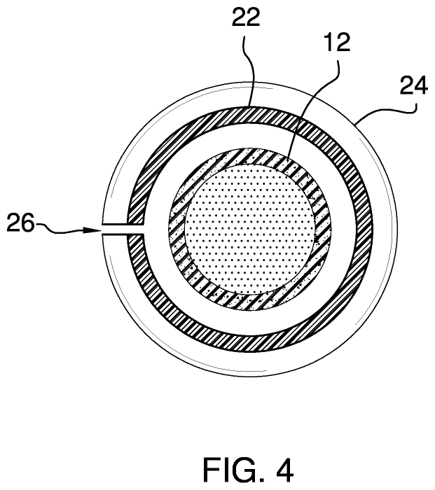

With reference now to the drawings, and in particular to FIGS. 1 through 4 thereof, a new hose device embodying the principles and concepts of an embodiment of the disclosure and generally designated by the reference numeral 10 will be described.

As best illustrated in FIGS. 1 through 4, the oxygen hose system 10 generally comprises an oxygen hose 12 that can be fluidly coupled to an oxygen source 14 for supplying breathing oxygen to a patient. The oxygen hose 12 may be medical grade oxygen hose and the oxygen source 14 may be a tank of pressurized gaseous oxygen. A sleeve 16 is provided and the sleeve 16 is positionable around the oxygen hose 12. The sleeve 16 is comprised of a resilient material to inhibit the oxygen hose 12 from being chewed by a domesticated animal, such as a dog or a cat. Additionally, the sleeve 16 is comprised of a flexible material thereby facilitating the oxygen hose 12 to be routed in various directions. The sleeve 16 is additionally comprised of an oxygen safe material that conforms to safety regulations that apply to supplying breathing oxygen.

The sleeve 16 has a first end 18, a second end 20 and an outer wall 22 extending therebetween, and the sleeve 16 is elongated between the first end 18 and the second end 20. The outer wall 22 has a plurality of ridges 24 each extending outwardly therefrom and each of the ridges 24 extends around a full circumference of the outer wall 22. The ridges 24 are spaced apart from each other and are distributed between the first end 18 and the second end 20. The outer wall 22 has a cut 26 extending between the first end 18 and the second end 20. The cut 26 is spreadable open to facilitate the sleeve 16 to be positioned around the oxygen hose 12.

In use, the cut 26 in the sleeve 16 is spread open to facilitate the sleeve 16 to be positioned around the oxygen hose 12. In this way sleeve 16 can be positioned around the oxygen hose 12 without having to route the oxygen hose 12 through the sleeve 16. The sleeve 16 protects the oxygen hose 12 from being chewed or scratched by a domesticated animal. Additionally, the sleeve 16 inhibits the oxygen hose 12 from becoming tangled and pinched thereby ensuring consistent flow through the oxygen hose 12.

With respect to the above description then, it is to be realized that the optimum dimensional relationships for the parts of an embodiment enabled by the disclosure, to include variations in size, materials, shape, form, function and manner of operation, system and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An oxygen hose system having enhanced durability and resistance to tangles, said system comprising:
an oxygen hose being configured to be fluidly coupled to an oxygen source for supplying breathing oxygen to a patient; and
a sleeve being positionable around said oxygen hose, said sleeve being comprised of a resilient material wherein said sleeve is configured to inhibit said oxygen hose from being chewed by a domesticated animal, said sleeve being comprised of a flexible material thereby facilitating said oxygen hose to be routed in various directions.

2. The system according to claim 1, wherein said sleeve has a first end, a second end and an outer wall extending therebetween, said sleeve being elongated between said first end and said second end, said outer wall having a plurality of ridges extending outwardly therefrom.

3. The system according to claim 2, wherein each of said ridges extends around a full circumference of said outer wall.

3. The system according to claim 2, wherein said ridges are spaced apart from each other and are distributed between said first end and said second end.

4. The system according to claim 2, wherein said outer wall has a cut extending between said first end and said second end, said cut being spreadable open to facilitate said sleeve to be positioned around said oxygen hose.

5. An oxygen hose system having enhanced durability and resistance to tangles, said system comprising:
an oxygen hose being configured to be fluidly coupled to an oxygen source for supplying breathing oxygen to a patient; and
a sleeve being positionable around said oxygen hose, said sleeve being comprised of a resilient material wherein said sleeve is configured to inhibit said oxygen hose from being chewed by a domesticated animal, said sleeve being comprised of a flexible material thereby facilitating said oxygen hose to be routed in various directions, said sleeve having a first end, a second end and an outer wall extending therebetween, said sleeve being elongated between said first end and said second end, said outer wall having a plurality of ridges extending outwardly therefrom, each of said ridges extending around a full circumference of said outer wall, said ridges being spaced apart from each other and being distributed between said first end and said second end, said outer wall having a cut extending between said first end and said second end, said cut being spreadable open to facilitate said sleeve to be positioned around said oxygen hose.

6. A method is protecting an oxygen hose from tangles and from abrasion, the steps of the method comprising:
providing an oxygen hose being configured to be fluidly coupled to an oxygen source for supplying oxygen for breathing;
providing a sleeve having a first end and a second end, said sleeve being comprised of a resilient material, said sleeve having a cut extending between said first end and said second end;
spreading said cut open; and
positioning said sleeve around said oxygen hose having said oxygen hose extending outwardly through said first end and said second end.
